(19) United States
(12) Patent Application Publication
(10) Pub. No.: US 2006/0240545 A1
(43) Pub. Date: Oct. 26, 2006

(54) FLOW CELL AND A REACTOR FOR REACTIONS BETWEEN FIXED REACTING SUBSTANCES AND LIQUID REACTING SUBSTANCES, AND A METHOD FOR OPERATING THE REACTOR

(75) Inventors: Maiko Tomida, Yokohama-shi (JP); Toru Myogadani, Yokohama-shi (JP)

Correspondence Address: DARBY & DARBY P.C., P. O. BOX 5257, NEW YORK, NY, 10150-5257, US

(73) Assignee: MORITEX CORPORATION, Shibuya-ku (JP)

(21) Appl. No.: 11/379,905

(22) Filed: Apr. 24, 2006

(30) Foreign Application Priority Data
Apr. 26, 2005 (JP) 2005-128632

(51) Int. Cl. C12M 3/00

(52) U.S. Cl. 435/289.1

(57) ABSTRACT

A flow cell having a first plate having a continuous groove formed as a passage on one surface of the plate and having gas/liquid inlet/outlet portions at both the ends of the continuous groove, and a second plate to be kept in contact with said one surface of the first plate, wherein at least either of the first and second plates allows light transmission; fixable reacting substances are fixed on the surface of the second plate, to be kept in contact with the first plate, at plural places corresponding to the continuous groove; and the first and second plates are kept in contact with each other to support each other, for establishing the passage of any one of liquid reacting substances common to the plural fixed reacting substances.

### INCORPORATION BY REFERENCE

The present application claims priority under 35 U.S.C. §119 to Japanese Patent Application No. 2005-128632 filed on Apr. 26, 2005. The content of the application is incorporated herein by reference in its entirety.

### FIELD OF THE INVENTION

The present invention relates to a flow cell and a reactor for reactions between fixed reacting substances and liquid reacting substances such as antigen-antibody reactions, and a method for operating the reactor.

### BACKGROUND OF THE INVENTION

Conventional techniques developed for measuring the amount of a test substance in a sample include immunoassay using antigen-antibody reactions.

One of immunoassay techniques is enzyme immunoassay (EIA) in which antigen-antibody reactions are traced using enzyme activity as a label, for determining the amount of an antigen or antibody based on the result. The EIA methods include chemiluminescent enzyme immunoassay (CLEIA) using chemiluminescence, and fluorescence immunoassay (FIA) using a fluorescent substance as a label.

The former method has a process in which a sample and an enzyme-labeled antibody are made to react with an antigen against a test substance carried as a solid phase on a carrier, and a chemiluminescent substrate is added to the reaction mixture. The substrate is decomposed by the enzyme to emit luminescence proportional to the amount of the enzyme, and the luminescent volume is measured for determination.

In the latter method, an antibody labeled with a fluorescent substance is made to react with a sample reacting with an antigen, and excitation light is irradiated. Then, the fluorescence volume emitted from the labeled substance is measured for determination.

As a further other determination method, it is shown in the non patent document, “Microarrays for the Screening of Allergen-Specific IgE in Human Serum” (Anal. Chem., 2003, 75(3), 556-562) that slides respectively having an antigen fixed are made to react with a sample and an enzyme-labeled antibody in this order, and chemiluminescent reactions are caused in a flow cell in which chips respectively having a groove for allowing a chemiluminescent substrate to flow in it are laminated, in order to measure the luminescent intensities.

As a still further other determination method, Japanese patent document 1, No. JP6-148075A describes that a reactor, in which two flat plates are laminated through two prismatic spacers disposed in parallel to and in opposite to each other at the edges of the two flat plates, to form a clearance between the two flat plates for allowing capillary injection and discharge, is used to perform an antigen-antibody reaction and a chemiluminescent reaction.

As a still further other determination method, Japanese patent document 2, No. JP2003-114229A describes a microchannel chip, in which a sample and a labeled antibody are made to react with each other beforehand, and a reaction is detected at the reaction site at which a substance specifically bound to a test substance is fixed.

However, the above-mentioned conventional techniques have the following problems.

First, the conventional CLEIA and FIA have a problem that it takes a relatively long time for sufficiently performing the antigen-antibody reactions.

Furthermore, the conventional technique described in said non patent document has a problem that since the reactions with a sample and an enzyme-labeled antibody must be performed in a separate process, the working efficiency is low. On the other hand, it can be considered to let the sample and the enzyme-labeled antibody flow in the flow cell. However, since the inner form of the flow cell is rhombic, the liquid is likely to spread, and mixing of liquids (contamination) is feared. Furthermore, there is a method for preventing the mixing of liquids, in which air is placed between the liquids when the liquids are allowed to flow in a pipe. However, since the inner form of the flow cell is rhombic, air is likely to deformed, and there also remains a fear of contamination. Moreover, the conventional technique has a problem that since the number of pumps is large the equipment is expensive and large.

Next, the conventional technique described in said patent document 1 has problems that since capillarity is used for liquid injection and discharge, the system is open, and therefore that automation is difficult and that the flow velocity of injection and discharge cannot be controlled.

Furthermore, the conventional technique described in said patent document 2 has problems that a large amount of a labeled antibody is necessary since it is necessary to perform the reaction between a sample and the labeled antibody beforehand, and that since the reaction site is small, the number of reactions detectable at a time is limited.

The present invention has been made in view of the above-mentioned problems. The object of this invention is to provide a flow cell which allows the reactions between fixed reacting substances and liquid reacting substances such as antigen-antibody reactions to be accomplished efficiently in a short time using a small amount of a sample without mixing it, which allows the amount of a test substance in a sample to be determined as in immunoassay, and which is simple to operate and can be used for antigen-antibody reactions. The object of this invention is also to provide a reactor allowing automatic operation.

### SUMMARY OF THE INVENTION

To solve the above-mentioned problems, at first, this invention is a flow cell for reactions between fixed reacting substances and liquid reacting substances, having a first plate having a continuous groove formed as a passage on one surface of the plate and having gas/liquid inlet/outlet portions at both the ends of the continuous groove, and a second plate to be kept in contact with said one surface of the first plate, wherein at least either of the first and second plates allows light transmission; fixable reacting substances are fixed on the surface of the second plate, to be kept in contact with the first plate, at plural places corresponding to the continuous groove; and the first and second plates are kept in contact with each other to support each other, for establishing the passage of any one of liquid reacting substances common to the plural fixed reacting substances.

Next, this invention is a flow cell for reactions between fixed reacting substances and liquid reacting substances, having a first plate having a continuous groove formed as a passage on one surface of the plate and having gas/liquid inlet/outlet portions at both the ends of the continuous groove, and a second plate to be kept in contact with said one surface of the first plate, wherein at least either of the first and second plates allows light transmission; fixable reacting substances are fixed on the first plate at plural places of the continuous groove; and the first and second plates are kept in contact with each other to support each other, for establishing the passage of any one of liquid reacting substances common to the plural fixed reacting substances.

Furthermore, this invention for the aforesaid flow cell for reactions between fixed reacting substances and liquid reacting substances, wherein the continuous groove extends in a zigzag line.

Still furthermore, this invention for the aforesaid flow cell for reactions between fixed reacting substances and liquid reacting substances, wherein the first and second plates are held between rigid support plates in such a manner that the first and second plates are kept in contact with each other and supported by the support plates, and the support plate corresponding to the first or second plate that allows light transmission has an opening formed in the portion corresponding to the range in which the fixable reacting substances are fixed or allows light transmission at least in the portion corresponding to the range in which the fixable reacting substances are fixed.

Still furthermore, the aforesaid flow cell for reactions between fixed reacting substances and liquid reacting substances, where the support plate has no opening formed and has a heating means installed in the portion corresponding to the range in which the fixable reacting substances are fixed.

Still furthermore, the fixable reacting substances can be antigens and the liquid reacting substances are a sample, a washing liquid, an enzyme-labeled substance, and a chemiluminescent substrate.

Still furthermore, reactions between fixed reacting substances and liquid reacting substances, has the aforesaid flow cell installed in a black box, a digital camera disposed on the light transmitting plate side, a gas/liquid supply pipe connected with one of the gas/liquid inlet/outlet portions of the first plate, and a waste liquor pipe connected with the other gas/liquid inlet/outlet portion, wherein the gas/liquid supply pipe is connected with a gas/liquid supply device consisting of a syringe pump, a selector valve and various liquid reacting substance containers.

Still furthermore, the aforesaid reactor for reactions between fixed reacting substances and liquid reacting substances, wherein the gas/liquid supply device has the syringe pump, a system liquid container disposed on the suction side of the syringe pump, the selector valve with its common port connected with the discharge side of the syringe pump through a common pipe having a sample loop, plural liquid reacting substance containers connected through pipes to respective selector ports of the selector valve, a waste liquor pipe connected to a selector port of the selector valve, a suction pipe connected to a selector port of the selector valve, and the common port of the selector valve connected with the gas/liquid supply pipe.

Still furthermore, the liquid reacting substance containers can be a washing liquid container, a sample container, an enzyme-labeled antibody container, and a chemiluminescent substrate container.

Still furthermore, a method for operating the aforesaid reactor for reactions between fixed reacting substances and liquid reacting substances, characterized in that when a system liquid discharged by the syringe pump is used to supply the liquid in any one of the liquid reacting substance containers to the flow cell through the gas/liquid supply pipe, air is sucked into the sample loop from the suction pipe through the selector valve before the liquid to be supplied is sucked, in order to let the air intervene between the liquid to be sucked next into the sample loop and the system liquid.

In the flow cell of this invention, the passage for letting fixed reacting substances and any one of liquid reacting substances react with each other can be established when the first plate having a continuous groove formed, for example, in zigzag and the second plate are kept in contact with each other for supporting each other. Therefore, fixable reacting substances can be easily fixed at plural places corresponding to the continuous groove while the plates are disconnected from each other, and the reactions between the liquid reacting substance flowing in the common passage and the plural fixed reacting substances can be performed efficiently. Furthermore, the luminescences generated by the reactions can be detected by a digital camera through the light transmitting first or second plate.

The liquid reacting substance flowing in the passage is kept in the passage established by the continuous groove and the plate surface and does not spread. So, if air is made to intervene between the liquid reacting substance and the system liquid when the liquid reacting substance is moved by using the system liquid, the interfaces consisting of liquid-air-liquid are maintained also in the passage. Therefore, the contamination between liquids can be reliably prevented.

Furthermore, since the first and second plates kept in contact with each other for supporting each other can be disconnected from each other, they can be repetitively used. Especially when the fixable reacting substances are fixed on the second plate, not on the first plate having a continuous groove formed, the first plate can also be used commonly to other second plates having different fixed reacting substances.

Next, this invention has the gas/liquid supply device for supplying the liquid reacting substance to the flow cell of this invention having a syringe pump, a system liquid container disposed on the suction side of the syringe pump, a selector valve with its common port connected with the discharge side of the syringe pump through a common pipe having a sample loop, plural liquid reacting substance containers connected through pipes to respective selector ports of the selector valve, a waste liquor pipe connected to a selector port of the selector valve, a suction pipe connected to a selector port of the selector valve, and the common port of the selector valve connected with the gas/liquid supply port.

Therefore, the number of pumps can be minimized to lower the cost and to save the space.

### BRIEF DESCRIPTION OF THE DRAWINGS

FIG. 1 is an exploded view showing the components of the flow cell of this invention as a first embodiment.

FIG. 2 is a side view showing the first and second plates kept in contact with each other for supporting each other.

FIG. 3 is a perspective view showing a portion of the first plate.

FIG. 4 is a transverse sectional view showing a portion of FIG. 2.

FIG. 5 is a longitudinal sectional view showing a portion of FIG. 2.

FIG. 6 is an exploded view showing the components of the flow cell of this invention in another embodiment.

FIG. 7 is an exploded view showing the components of the flow cell of this invention in a further embodiment.

FIG. 8 is a vertical sectional view showing the support state of the flow cell of this invention in the embodiment of FIG. 7.

FIG. 9 is an exploded view showing the components of the flow cell of this invention as another embodiment.

FIG. 10 is a vertical sectional view showing the support state of the flow cell of this invention in the embodiment of FIG. 9.

FIG. 11 is an exploded view showing the components of the flow cell of this invention in a further embodiment.

FIG. 12 is a system illustration showing the entire constitution of the reactor using the flow cell of this invention.

FIG. 13 is an expanded view showing a portion of FIG. 12.

FIG. 14 is a flow chart showing the operation procedure in which the reactor of this invention is used to determine antigen-antibody reactions according to CLEIA.

### DETAILED DESCRIPTION OF THE INVENTION

This invention is explained below in detail in reference to the drawings showing embodiments.

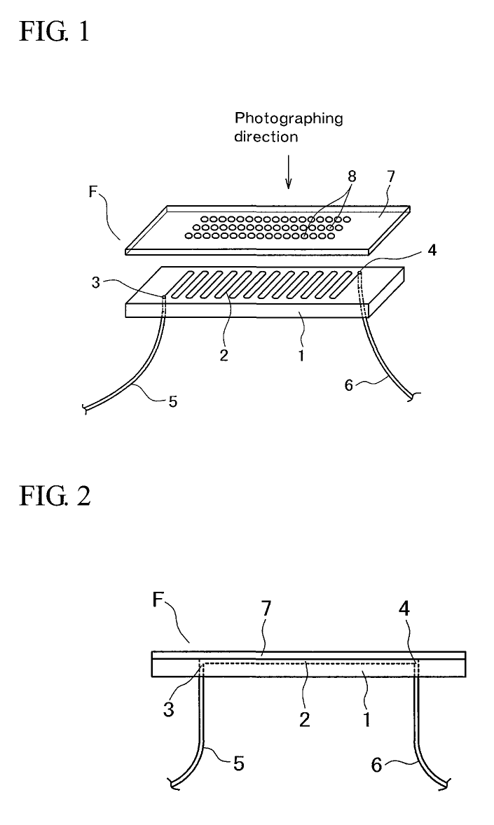

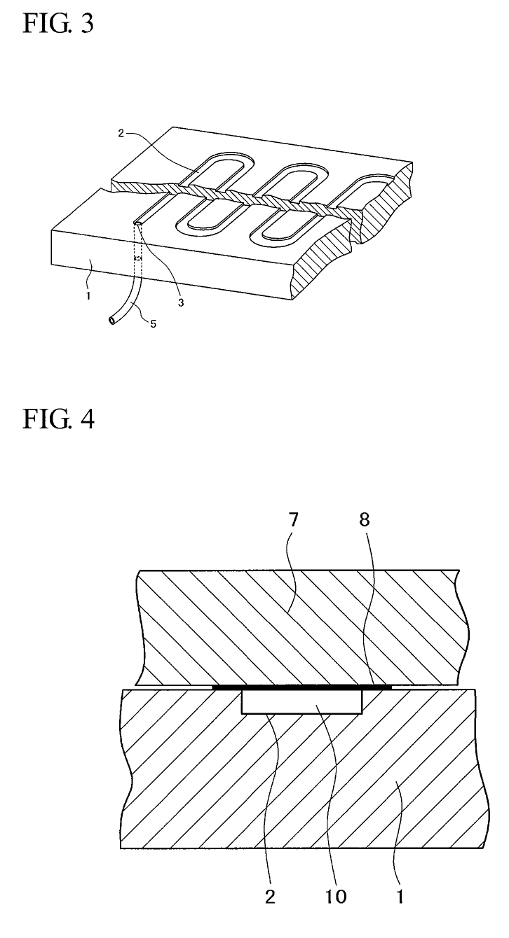

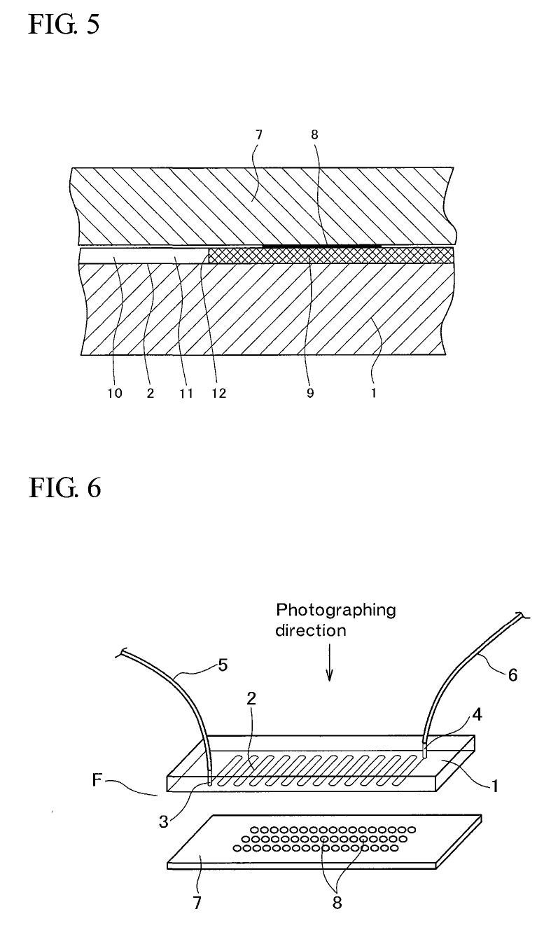

FIGS. 1 to 5 show an embodiment of the flow cell F of this invention. Symbol 1 denotes a first plate, and on one surface of the first plate 1, i.e., on the upper surface in the drawings, a continuous groove 2 for forming a passage is formed, and gas/liquid inlet/outlet portions 3 and 4 are provided at both the ends of the continuous groove 2. Pipes are connected with the gas/liquid inlet/outlet portions 3 and 4. One of the pipes is a gas/liquid supply pipe 5 connected with the gas/liquid supply device described later, and the other pipe is a waste liquor pipe 6. Meanwhile, in the drawings, the continuous groove 2 is formed zigzag in the longitudinal direction of the first plate 1, but it can also be formed zigzag in the transverse direction. As the case may be, the groove can also be formed spirally.

Symbol 7 denotes a second plate, and the second plate 7 allows light transmission. The lower surface of the second plate 7 in the drawings contacts the upper surface of the first plate 1. The second plate 7 has fixable reacting substances 8 fixed by a predetermined carrier at plural places corresponding to the continuous groove 2, on the surface to be kept in contact with the first plate, i.e., on the lower surface in the drawings. In the drawings, the fixable reacting substances 8 are fixed at positions regularly arranged in lengthwise and crosswise directions like a matrix, to correspond to the zigzag continuous groove 2.

In the above constitution, if the first plate 1 and the second plate 7 are kept in contact with each other for supporting each other, a passage 10 of a liquid reacting substance 9 common to the fixable reacting substances 8 fixed at plural places can be established. The first plate 1 and the second plate 7 can be kept in contact with each other for supporting each other by using an adequate holding mechanism such as clamping devices.

In the above constitution, the liquid reacting substance 9 flowing in the gas/liquid supply pipe 5 from the gas/liquid supply device described later goes through the gas/liquid inlet/outlet portion 3 on one side to reach the passage 10 established by the continuous groove 2 and the surface of the second plate 7. While flowing zigzag in the passage 10, the liquid reacting substance 9 reaches the plural positions of the fixed reacting substances 8 one after another, to cause reactions at those positions. The liquid reacting substance 9 can be passed from the gas/liquid inlet/outlet portion 3 on one side to the gas/liquid inlet/outlet portion 4 on the other side once only and discharged from the gas/liquid inlet/outlet portion 4 on the other side through the waste liquor pipe 6. However, if the liquid reacting substance 9 is reciprocated plural times between one side and the other side of the passage 10, reliability can be enhanced.

The liquid reacting substance 9 flowing in the passage 10 is kept within the passage 10 established by the continuous groove 2 and the plate surface and does not spread. So, as described later, if air 11 is made to intervene between the liquid reacting substance 9 and the system liquid when the liquid reacting substance 9 is moved by using the system liquid, the interfaces 12 of liquid-air-liquid are maintained also in the passage 10. So, the contamination between liquids can be reliably prevented.

As described above, in the case where the flow cell F of this invention is used, since the first plate 1 having the continuous groove 2 formed, for embodiment, zigzag and the second plate 2 are kept in contact with each other for supporting each other to establish the passage 10 for allowing the fixed reacting substances 8 and the liquid reacting substance 9 to react with each other, the fixable reacting substances 8 can be easily fixed at plural places corresponding to the continuous groove 2 while the first plate 1 and the second plate 7 are disconnected from each other. Furthermore, the reactions between the liquid reacting substance 9 flowing in the common passage 10 and the plural fixable reacting substances 8 fixed along the passage 10 can be performed efficiently. Moreover, the luminescences generated by the reactions can be photographed by a digital camera from above in the drawing through the light transmitting plate, i.e., the second plate 7 in this case, and it can be detected using a computer, etc.

Furthermore, since the first plate 1 and the second plate 7 kept in contact with each other for supporting each other can be disconnected from each other, they can be repetitively used. Especially if the fixable reacting substances 8 are fixed on the second plate 2, not on the first plate 1 having the continuous groove 2 formed, as in this embodiment, the first plate 1 can also be used commonly to other second plates 7 having different fixable reacting substances 8 fixed.

However, as another embodiment, the fixable reacting substances 8 can also be fixed on the first plate 1 having the continuous groove 2 formed, at plural places along the continuous groove 2.

In the above-mentioned embodiment, the second plate 7 allows light transmission, and the luminescences generated by reactions are photographed by a digital camera for detection from the second plate 7 side. However, as described in the following embodiment, the first plate 1 can be made to allow light transmission for light detection from the first plate 1 side.

FIG. 6 shows a further embodiment of the flow cell F of this invention.

In this embodiment, the other plate is made to allow light transmission, and the photographing by a digital camera is performed in the other direction, contrary to embodiment 1. Since the other components are the same as those of embodiment 1, the same symbols are used to denote the components corresponding to those of embodiment 1, for avoiding double explanation.

That is, in this embodiment, the first plate 1 having the zigzag continuous groove 2 formed is made to allow light transmission, and the second plate 7 is made not to allow light transmission. On the other hand, since the digital camera is disposed above the flow cell F as in embodiment 1, the first plate 1 is disposed above, and the second plate 7 is disposed below.

The action of the flow cell F of embodiment 2 is the same as that of the embodiment above and is obvious. So, double explanation is avoided.

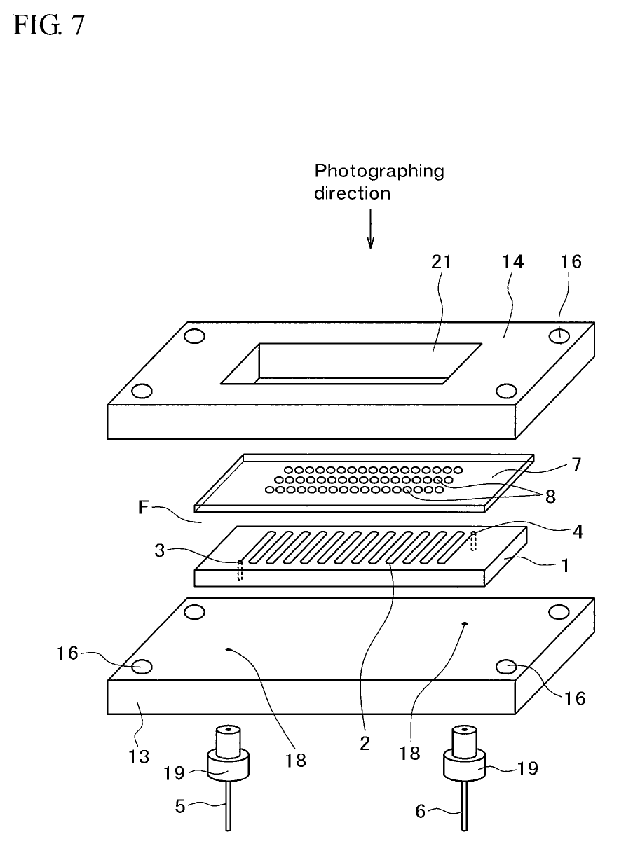

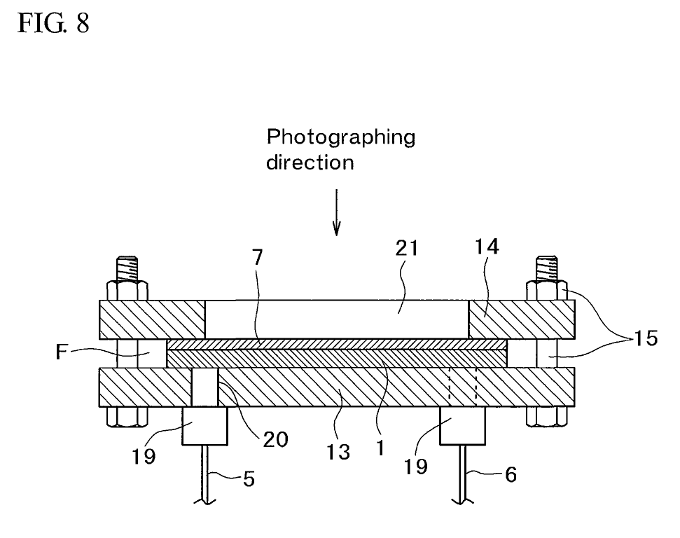

FIGS. 7 and 8 show another embodiment of the flow cell F of this invention. This embodiment show a particular support mechanism for keeping the first and second plates in contact with each other in the flow cell F shown in embodiment 1. The same symbols are used to denote the components corresponding to those of embodiment 1, for avoiding double explanation.

That is, in this embodiment, a first support plate 13 is provided in correspondence to the first plate 1 having the continuous groove 2 formed, and a second support plate 14 is provided in correspondence to the light transmitting second plate 7.

The first support plate 13 and the second support plate 14 are blocks made of a rigid material such as aluminum, and they have through holes 16 formed for clamping parts 15 such as bolts and nuts shown in FIG. 8. Furthermore, the first support plate 13 has through holes 18 formed at the positions corresponding to the gas/liquid inlet/outlet portions 3 and 4 of the first plate 1, and has connecting portions 20 to be connected with the connecting members 19 provided at the ends of the gas/liquid supply pipe 5 and the waste liquor pipe 6 by threaded engagement, etc. Moreover, the second support plate 14 has an opening 21 formed in correspondence to the fixing range of the fixable reacting substances 8 fixed on the light transmitting second plate 7.

In the above constitution, the first plate 1 and the second plate 7 kept in contact with each other are held between the first support plate 13 and the second support plate 14, and the bolts and nuts 15 are tightened through the through holes 16, to support the first plate 1 and the second plate 7 kept in contact with each other. Meanwhile, any other adequate holding mechanism using a clamp mechanism can also be used instead of the bolts and nuts 15 for the first support plate 13 and the second support plate 14.

In the aforesaid state, the connecting members 19 provided at the ends of the gas/liquid supply pipe 5 and the waste liquor pipe 6 can be connected with the connecting portions 20 by threaded engagement, etc., and the liquid reacting substance 9 can be supplied from the gas/liquid supply pipe 5 to the passage 10 of the flow cell F, and be made to react with the plural fixable reacting substances 8 fixed along the passage 10, then being discharged from the waste liquor pipe 6.

The luminescences generated by reactions can be photographed by a digital camera through the light transmitting plate, the second plate 7 in this case and the opening 21 of the second support plate 14 from above in the drawing, for detection.

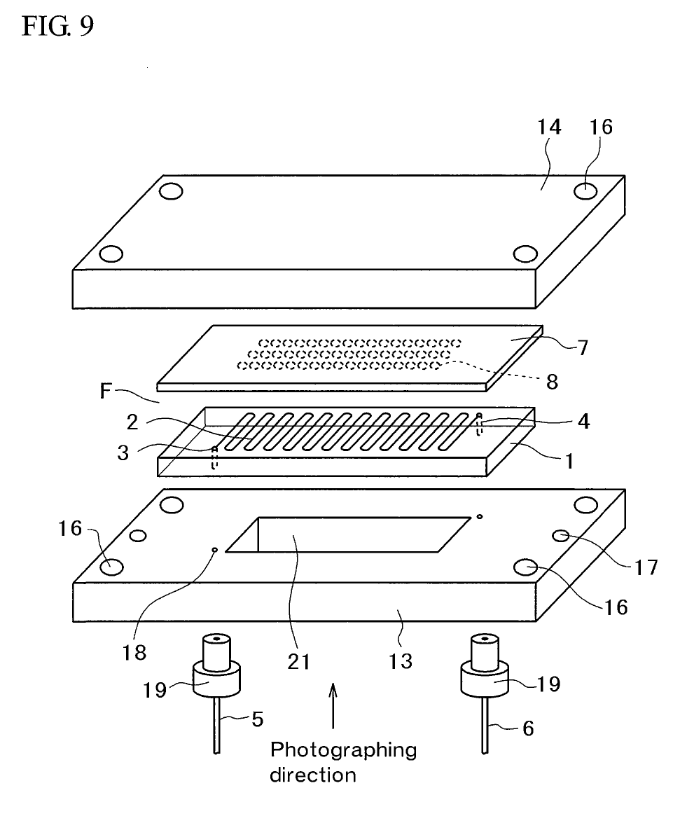

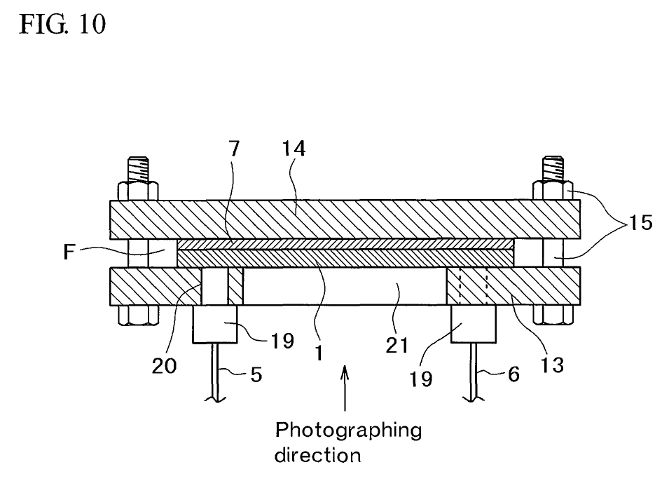

FIGS. 9 and 10 show a further embodiment of the flow cell F of this invention. This embodiment shows a particular support mechanism for the first and second plates kept in contact with each other of the flow cell F shown in embodiment 2, and the same symbols are used to denote the components corresponding to those of the previous embodiments, for avoiding double explanation.

That is, in this embodiment, a first support plate 13 is provided in correspondence to the first plate 1 having the continuous groove 2 and a second support plate 14 is provided in correspondence to the second plate 7 as in embodiment 3. However, since the first plate 1 allows light transmission, an opening 21 corresponding to the fixing range of the fixable reacting substances 8 fixed on the second plate 7 is formed in the first support plate 13, and the photographing by a digital camera is performed in the upward direction. The other components are the same as those of the embodiment above and the action is obvious. So, the same symbols are used to denote the same components as those above, for avoiding double explanation.

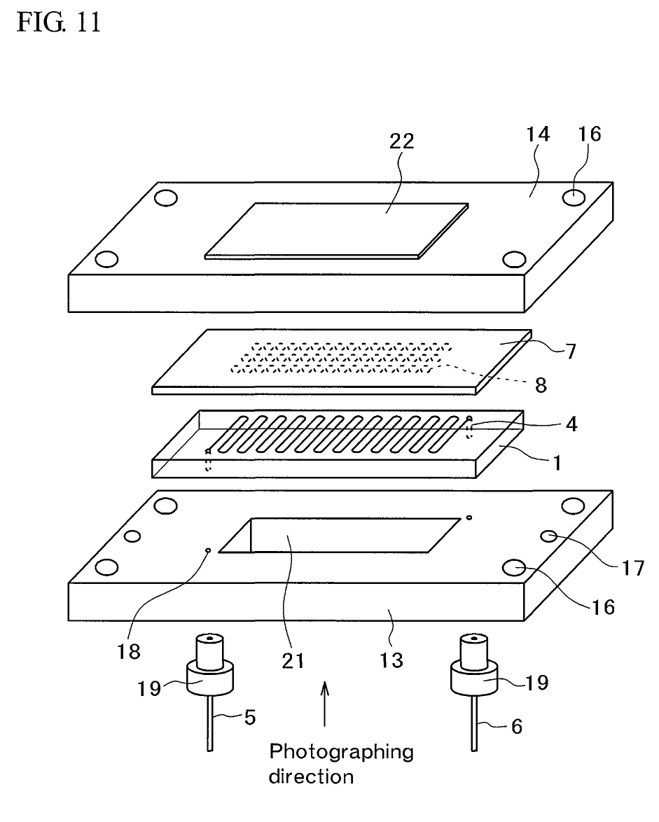

FIG. 11 shows another embodiment of the flow cell F of this invention. In this embodiment, a heater 22 is installed on the second support plate 14 in the constitution of embodiment 4, so that the temperature can be controlled when the fixed reacting substances 8 and the liquid reacting substance 9 are made to react with each other in the flow cell F. The other components are the same as those above, and the action is obvious. So, the same symbols are used to denote the same components as those of the above embodiment for avoiding double explanation.

Meanwhile, as another embodiment, a temperature controller capable of heating and cooling can also be installed instead of the heater 22.

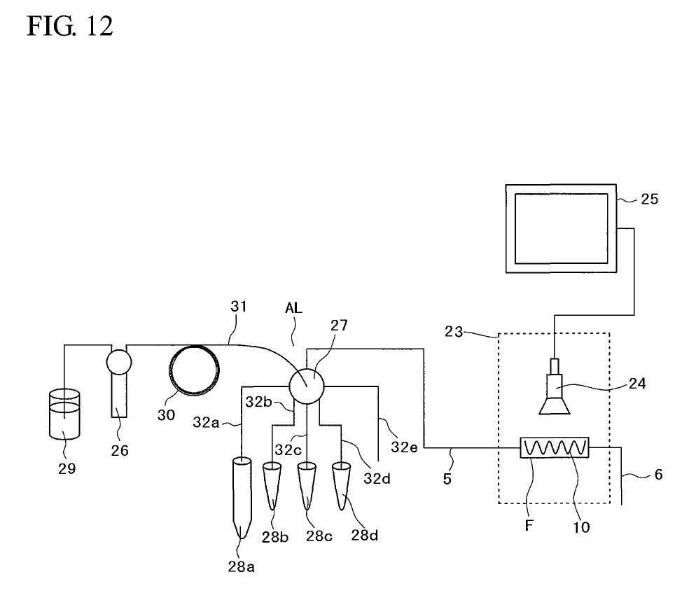

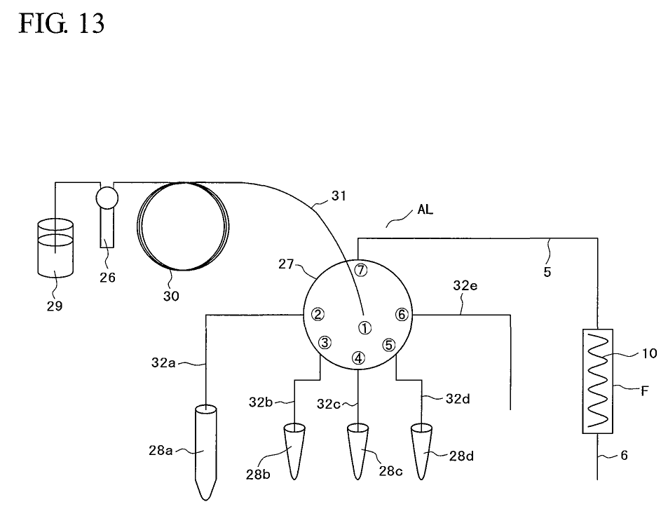

FIG. 12 is a system illustration showing the entire constitution of the reactor using the flow cell F of this invention, and FIG. 13 is an expanded view showing a portion of FIG. 12.

Symbol 23 denotes a black box, and the flow cell F is installed in the black box 23. Above the flow cell F, a digital camera 24 using CCD and C-MOS sensor is installed. The output of the camera is applied to the input of a computer 25.

Furthermore, the gas/liquid supply pipe 5 is connected with the air/liquid inlet/outlet portion 3 on one side of the first plate 1 of the flow cell F, and the waste liquor pipe 6 is connected with the gas/liquid inlet/outlet portion 4 on the other side. The waste liquor pipe 6 is extended to an adequate discharge place, and the gas/liquid supply pipe 5 is connected with a gas/liquid supply device AL consisting of a syringe pump 26, a selector valve 27 and various liquid reacting substance containers 28 (28a, 28b, 28c, 28d, . . . ).

The gas/liquid supply device AL will be explained further in detail. The gas/liquid supply device AL consists of the syringe pump 26, a system liquid container 29 disposed on the suction side of the syringe pump 26, the selector valve 27 with its common port (1) connected with the discharge side of the syringe pump 26 through a common pipe 31 having a sample loop 30, the plural liquid reacting substance containers 28 (28a, 28b, 28c, 28d, . . . ) respectively connected through pipes 32 (32a, 32b, 32c, 32d, . . . ) to selector ports (2) to (5) of the selector valve 27, an exhaust and suction pipe 32e connected to a selector port (6) of the selector valve 27, and a selector port (7) of the selector valve 27 connected to the gas/liquid supply pipe 5.

In the case where the reactor with the above constitution is used as a reactor for antigen-antibody reactions, among the liquid reacting substance containers 28, 28a denotes a washing liquid container; 28b, a sample container; 28c, an enzyme-labeled antibody container; and 28d, a chemiluminescent substrate container.

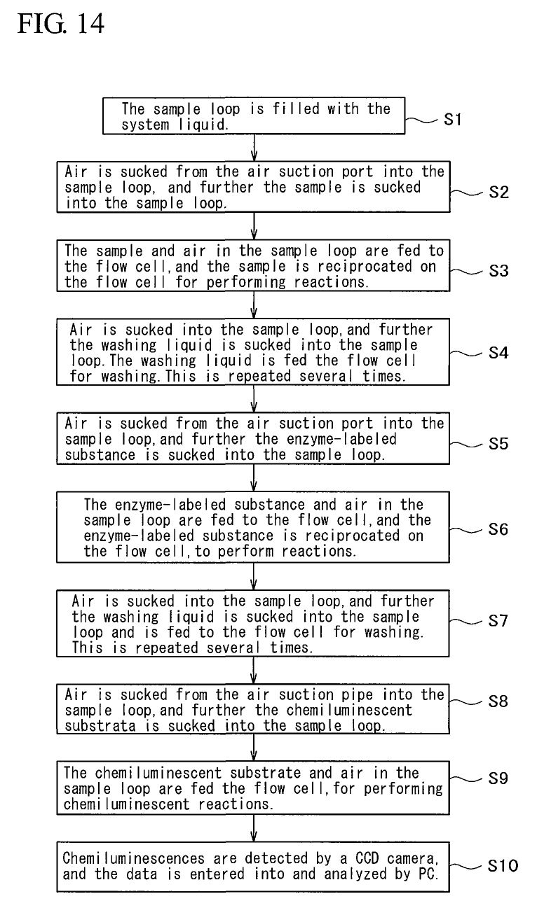

In the above constitution, the operation procedure of the reactor as an antigen-antibody reactor will be explained in reference to the flow chart of FIG. 14.

At first, at step S1, the syringe pump 26 is operated regularly to fill the sample loop 30 with the system liquid sucked from the system liquid container 29. In this case, the selector port (6) is selected at the selector valve 27, so that the air in the sample loop 30 is discharged into open air through the pipe 32e.

Then at step S2, the syringe pump 26 is operated reversely to suck air into the sample loop 30, and subsequently the selector port (3) is selected at the selector valve 27, to suck the sample from the sample container 28b into the sample loop 30.

At this point of time, the sample and the system liquid are kept away from each other owing to the intervening air in the sample loop 30, and no contamination occurs.

Then at step S3, the selector port (7) is selected at the selector valve 27, and the syringe pump 26 is operated regularly. With this operation, the sample in the sample loop 30 is pressed by the system liquid through air, to flow through the gas/liquid supply pipe 5, reaching the flow cell F. It flows in the passage 10 of the flow cell F, and reacts with the fixed reacting substances 8.

In this state, if the syringe pump 26 is operated in regular and reverse directions alternately plural times, the sample is reciprocated between one side and the other side of the passage 10 plural times, to enhance the reliability.

Then at step S4, the selector port (6) is selected at the selector valve 27, and the syringe pump 26 is operated reversely to suck air into the sample loop 30. Then, the selector port (2) is selected at the selector valve 27, to suck the washing liquid from the washing liquid container 28a into the sample loop 30.

Then the selector port (7) is selected at the selector valve 27, and the syringe pump 26 is operated regularly. With this operation, the washing liquid in the sample loop 30 is pressed by the system liquid through air and flows through the gas/liquid supply port 5, to reach the flow cell F. It flows in the passage 10 of the flow cell F and washes away the extra sample in the passage 10. Also in this case, if the syringe pump 26 is operated in regular and reverse directions alternately plural times, the washing liquid can be reciprocated between one side and the other side of the passage 10 plural times.

Then at step S5, the selector port (6) is selected at the selector valve 27, and the syringe pump 26 is operated reversely to suck air into the sample loop 30. Then the selector port (4) is selected at the selector valve 27, to suck the enzyme-labeled antibody from the enzyme-labeled antibody container 28c into the sample loop 30.

Then at step S6, the selector port (7) is selected at the selector valve 27, and the syringe pump 26 is operated regularly. With this operation, the enzyme-labeled antibody in the sample loop 30 is pressed by the system liquid through air and flows through the gas/liquid supply pipe 5, to reach the flow cell F, and it flows in the passage 10 of the flow cell F and reacts with the fixed reacting substances 8. Also in this case, if the syringe pump 26 is operated in the regular and reverse directions alternately plural times, the enzyme-labeled antibody can be reciprocated between one side and the other side of the passage 10 plural times. Meanwhile, the washing liquid of the previous step is pressed by the system liquid remaining in the previous step and discharged through the waste liquor pipe 6.

Then at step S7, like step S4, the selector port (6) is selected at the selector valve 27, and the syringe pump 26 is operated reversely to suck air into the sample loop 30. Then the selector port (2) is selected at the selector valve 27, to suck the washing liquid from the washing liquid container 28a into the sample loop 30. Then the selector port (7) is selected at the selector valve 27, and the syringe pump 26 is operated regularly. With this operation, the washing liquid in the sample loop 30 is pressed by the system liquid through air and flows through the gas/liquid supply pipe 5, to reach the flow cell F, and it flows in the passage 10 of the flow cell F and washes away the enzyme-labeled antibody in the passage 10. Also in this case, if the syringe pump 26 is operated in the regular and reverse directions alternately plural times, the washing liquid can be reciprocated from one side to the other side of the passage 10 plural times.

Then at step S8, at first the selector port (6) is selected at the selector valve 27, and the syringe pump 26 is operated reversely to suck air into the sample loop 30, and then the selector port (5) is selected at the selector valve 27, to suck the chemiluminescent substrate from the chemiluminescent substrate container 28d into the sample loop 30.

Then at step S9, the selector port (7) is selected at the selector valve 27, and the syringe pump 26 is operated regularly. With this operation, the chemiluminescent substrate in the sample loop 30 is pressed by the system liquid through air and flows through the gas/liquid supply pipe 5, to reach the flow cell F, and it flows in the passage 10 of the flow cell F and causes chemiluminescent reactions with the fixed reacting substances 8. In this case, to observe the respective luminescences of the plural fixed reacting substances, the chemiluminescent substance is fed till it fills the passage 10 of the flow cell F, and subsequently, the feed is stopped to let the chemiluminescent substance remain in the passage 10 of the flow cell F.

Then at step S10, the chemiluminescences from the positions of the fixed reacting substances 8 after lapse of these steps are photographed, and the luminescence data are fed as digital signals into the computer 25 and analyzed in the computer 25. The analyzed data are delivered from the computer 25 as predetermined.

Meanwhile in the above embodiments, air intervenes between the system liquid and each of the liquid reacting substances, but instead of air, an inert gas such as nitrogen gas can also be made to intervene. In this case, an inert gas vessel is connected, instead of a pipe, to the corresponding selector port of the selector valve 27, and the operation as described above can be performed. Furthermore, an insoluble liquid incapable of dissolving the system liquid and the various liquid reacting substances can also be made to intervene instead of the gas. In this case, an insoluble liquid container is disposed through a pipe connected to the corresponding selector port of the selector valve 27, and the same operation as described above can be performed. Moreover, if a system liquid that cannot dissolve any of the various liquid reacting substances is used, operation can be performed without letting any substance intervene.

In the above embodiments, an operation procedure for antigen-antibody reactions by CLEIA is shown, but the flow cell and the reactor of this invention can also be applied to FIA. In this case, an excitation light source can be installed in the black box 23.

Furthermore, the flow cell and the reactor of this invention can be applied not only to antigen-antibody reactions, but also to various reactions in which a liquid substance sampled from an organism or further chemically treated or chemically modified is made to react with various fixed proteins (cells or viruses), nucleic acids, cDNAs, DNAs, RNAs or the like capable of being specifically bound to a substance derived from an organism and having known base sequences, base lengths, chemical compositions, etc., for photochemical detection, to analyze the substance derived from an organism.

### EXAMPLE 1

A particular embodiment of the flow cell and the reactor of this invention, and a particular example of the antigen-antibody reactions performed using them are described below.

As the second plate 7 used as a component of the flow cell F, a light transmitting acrylic plate was used. As the first plate 1, a chip made of polydimethylsiloxane (PDMS) was used, and a zigzag continuous groove 2 with a groove width of 1 mm and a groove depth of 0.2 mm was formed in it. The first plate 1 and the second plate 7 were kept in contact with each other to form a flow cell. In this case, the inner capacity of the passage 10 was 75 μL.

On the other hand, antigens were fixed on the acrylic plate used as the second plate 7 by an optical fixing method.

The first plate 1 and the second plate 7 were not made to adhere to each other, but aluminum blocks as first and second plates as shown in FIG. 7 were used for contact bonding. A heater was installed on the aluminum block used as the first support plate, and antigen-antibody reactions were made to take place at a constant temperature (37 degrees Celsius). As the antigens, mite allergens were fixed, and serum was used as the sample. As the enzyme-labeled antibody, an antibody labeled with peroxidase was used.

Each liquid was fed by the gas/liquid supply device to the flow cell at a flow velocity of 3.3 μL/sec.

The antigen-antibody reactions were performed according to the procedure of FIG. 14.

As described above, the antigen-antibody reactions between fixed mite allergens and serum were confirmed using the reactor of this invention according to CLEIA. Furthermore, the amount of the antibody contained in the sample was determined.

### INDUSTRIAL APPLICABILITY

This invention as described above has the following features and is highly industrially applicable. In the flow cell of this invention, the passage in which fixed reacting substances and a liquid reacting substance are made to react with each other is established between a first plate having a continuous groove formed zigzag or the like and a second plate with both the plates kept in contact with each other for supporting each other. So, the fixable reacting substances can be easily fixed at plural places corresponding to the continuous groove while the first and second plates are disconnected from each other, and the many reactions between the liquid reacting substance flowing in the common passage and the plural fixable reacting materials fixed along the passage can be performed efficiently at a time. Furthermore, the luminescences generated by the reactions can be detected by a digital camera through the light transmitting first or second plate.

The liquid reactive substance flowing in the passage can be kept in the passage established by the continuous groove and the plate surface and does not spread. So, if air is made to intervene between the liquid reacting substance and a system liquid when the liquid reacting substance is moved by using the system liquid, interfaces of liquid-air-liquid can be maintained also in the passage, and the contamination between the liquids can be reliably prevented.

Since the first and second plates kept in contact with each other for supporting each other can be disconnected from each other, they can be repetitively used. Especially in the case where the fixable reacting substances are fixed on the second plate, not on the first plate having the continuous groove formed, the first plate can be used commonly also to other second plates having different fixable reacting substances fixed.

The gas/liquid supply device for supplying any of liquid reacting substances to the flow cell of this invention consists of a syringe pump, a system liquid container disposed on the suction side of the syringe pump, a selector valve with its common port connected with the discharge side of the syringe pump through a common pipe having a sample loop, plural liquid reacting substance containers connected through pipes to respective selector ports of the selector valve, a waste liquor pipe connected to a selector port of the selector valve, a suction pipe connected to a selector port of the selector valve, and the common port of the selector valve connected with the gas/liquid supply pipe. Therefore, the number of pumps can be minimized to lower the cost and to save the space.

Since the syringe pump used can be operated for sucking and discharging, any one of the liquid reacting substances such as a sample and a labeled substance can be reciprocated in the passage of the flow cell where reactions take place, to raise the reaction efficiency.

What is claimed is:

1. A flow cell for reactions between fixed reacting substances and liquid reacting substances, comprising a first plate having a continuous groove formed as a passage on one surface of the plate and having gas/liquid inlet/outlet portions at both the ends of the continuous groove, and a second plate to be kept in contact with said one surface of the first plate, wherein at least either of the first and second plates allows light transmission; fixable reacting substances are fixed on the surface of the second plate, to be kept in contact with the first plate, at plural places corresponding to the continuous groove; and the first and second plates are kept in contact with each other to support each other, for establishing the passage of any one of liquid reacting substances common to the plural fixed reacting substances.

2. A flow cell for reactions between fixed reacting substances and liquid reacting substances, comprising a first plate having a continuous groove formed as a passage on one surface of the plate and having gas/liquid inlet/outlet portions at both the ends of the continuous groove, and a second plate to be kept in contact with said one surface of the first plate, wherein at least either of the first and second plates allows light transmission; fixable reacting substances are fixed on the first plate at plural places of the continuous groove; and the first and second plates are kept in contact with each other to support each other, for establishing the passage of any one of liquid reacting substances common to the plural fixed reacting substances.

3. A flow cell for reactions between fixed reacting substances and liquid reacting substances, according to claim 1, wherein the continuous groove extends in a zigzag line.

4. A flow cell for reactions between fixed reacting substances and liquid reacting substances, according to claim 1, wherein the first and second plates are held between rigid support plates in such a manner that the first and second plates are kept in contact with each other and supported by the support plates, and the support plate corresponding to the first or second plate that allows light transmission has an opening formed in the portion corresponding to the range in which the fixable reacting substances are fixed or allows light transmission at least in the portion corresponding to the range in which the fixable reacting substances are fixed.

5. A flow cell for reactions between fixed reacting substances and liquid reacting substances, according to claim 1, wherein the first and second plates are held between rigid support plates in such a manner that the first and second plates are kept in contact with each other and supported by the support plates, and the support plate corresponding to the first or second plate that allows light transmission allows light transmission at least in the portion corresponding to the range in which the fixable reacting substances are fixed.

6. A flow cell for reactions between fixed reacting substances and liquid reacting substances, according to claim 4, wherein the support plate having no opening formed has a heating means installed in the portion corresponding to the range in which the fixable reacting substances are fixed.

7. A flow cell for reactions between fixed reacting substances and liquid reacting substances, according to claim 1, wherein the fixable reacting substances are antigens and the liquid reacting substances are at least one of a sample, a washing liquid, an enzyme-labeled substance, and a chemiluminescent substrate.

8. A reactor for reactions between fixed reacting substances and liquid reacting substances, comprising the flow cell as set forth in claim 1 installed in a black box, a digital camera disposed on the light transmitting plate side, a gas/liquid supply pipe connected with one of the gas/liquid inlet/outlet portions of the first plate, and a waste liquor pipe connected with the other gas/liquid inlet/outlet portion, wherein the gas/liquid supply pipe is connected with a gas/liquid supply device consisting of a syringe pump, a selector valve, and various liquid reacting substance containers.

9. A reactor for reactions between fixed reacting substances and liquid reacting substances, according to claim 8, wherein the gas/liquid supply device consists of the syringe pump, a system liquid container disposed on the suction side of the syringe pump, the selector valve with its common port connected with the discharge side of the syringe pump through a common pipe having a sample loop, plural liquid reacting substance containers connected through pipes to respective selector ports of the selector valve, a waste liquor pipe connected to a selector port of the selector valve, a suction pipe connected to a selector port of the selector valve, and the common port of the selector valve connected with the gas/liquid supply pipe.

10. A reactor for reactions between fixed reacting substances and liquid reacting substances, according to claim 9, wherein the liquid reacting substance containers are at least one of a washing liquid container, a sample container, an enzyme-labeled antibody container, and a chemiluminescent substrate container.

11. A method for operating the reactor for reactions between fixed reacting substances and liquid reacting substances as set forth in claim 8 comprising the steps of:
discharging a system liquid by the syringe pump;
supplying the liquid in any one of the liquid reacting substance containers to the flow cell through the gas/liquid supply pipe;
sucking air into the sample loop from the suction pipe through the selector valve before the liquid to be supplied is sucked;
intervening the air between the liquid to be sucked next into the sample loop and the system liquid.

12. A method for operating the reactor for reactions between fixed reacting substances and liquid reacting substances as set forth in claim 8 comprising the steps of:
discharging a system liquid by the syringe pump;
supplying the liquid in any one of the liquid reacting substance containers to the flow cell through the gas/liquid supply pipe;
sucking an inert gas into the sample loop through the selector valve from a pipe connected to an inert gas vessel before the liquid to be supplied is sucked; and
intervening the inert gas intervene between the liquid to be sucked next into the sample loop and the system liquid.

13. A method for operating the reactor for reactions between fixed reacting substances and liquid reacting substances as set forth in claim 8 comprising the steps of:
discharging a system liquid by the syringe pump if used to supply the liquid in any one of the liquid reacting substance containers to the flow cell through the gas/liquid supply pipe, an insoluble liquid incapable of dissolving the system liquid and the various liquid reacting substances is sucked into the sample loop through the selector valve from a pipe connected to an insoluble liquid container before the liquid to be supplied is sucked, in order to let the insoluble liquid intervene between the liquid to be sucked next into the sample loop and the system liquid.
